(19) United States
(12) Patent Application Publication
(10) Pub. No.: US 2002/0168424 A1
(43) Pub. Date: Nov. 14, 2002

(54) Nitric oxide (NO) donor+cGMP-PDE5 inhibitor as a topical drug for glaucoma

(75) Inventors: Mohsen Shahinpoor, Albuquerque, NM (US); David Soltanpour, New York, NY (US); Parsa Shahinpoor, Albuquerque, NM (US)

Correspondence Address: MOHSEN SHAHINPOOR, 909 VIRGINIA, NE, SUITE 205, ALBERQUERQUE, NM, 87108, US

(73) Assignee: Dr. Mohsen Shahinpoor, Albuquerque, NM

(21) Appl. No.: 10/064,627

(22) Filed: Jul. 31, 2002

(51) Int. Cl. A61K 33/00; A61K 38/16; A61K 31/715; A61K 31/495; A61K 31/21

(52) U.S. Cl. 424/718; 514/252.12; 514/42; 514/509; 514/2

(57) ABSTRACT

A new topical drug (ointment or eye drop) for treating glaucoma or ocular hypertension in a patient, which comprises a mixture of a nitric oxide (NO) donor such as nitrovasodilators like minoxidil, nitroglycerin, L-arginine, isosorbide dinitrate, or nitroprusside, and a cyclic guanosine 3′,5′-monophosphate (cGMP) specific phosphodiesterase type 5 (PDE5) inhibitor such as sildenafil citrate (Viagra^{RTM}) in an ophthalmologically acceptable solution mix. In this manner there will be increased blood circulation to the optic nerve and the ocular hypotensive effect of the combined compounds is enhanced synergistically.

### BACKGROUND OF INVENTION

The Present invention provides a topical method of treating ocular hypertension or glaucoma. Particularly, the invention provides a method, which can synergistically enhance the aqueous humor outflow and the blood circulation to the optic nerve.

“Glaucoma” is a debilitating eye disease that is the leading cause of preventable blindness in the world. Primary Open Angle Glaucoma is the most common form of glaucoma. The disease is currently believed by experts to be mostly caused by the degeneration of the trabecular meshwork, leading to obstruction of the normal ability of aqueous humor to drain out of the anterior chamber of the eye (see, Vaughn, D. et al., General Ophthalmology, Appleton & Lange, Norwalk, Conn., pp. 213-230 (1992)). This reduced drainage of aqueous humor creates increased intraocular pressure (“IOP”), resulting in progressive visual loss and blindness if not treated in a timely fashion.

Aqueous humor is formed in the posterior chamber of the eye by the ciliary body and processes at a rate of approximately 2.5 microliters per minute, which then passes around the lens, through the pupillary opening in the iris and into the anterior chamber of the eye. Once in the anterior chamber, the aqueous humor drains out of the eye through two different routes. In the “uveoscleral” route, the fluid percolates between the muscle fibers of the ciliary body. This route accounts for approximately 15 to 20 percent of the aqueous outflow in human eye. However, the primary pathway for aqueous outflow in human eye is through the “canalicular” route that involves the trabecular meshwork and the Schlemm's canal. The trabecular meshwork is a ring of tissue, which is wedge-shaped in cross-section that runs around the circumference of the eye. It is composed of collagen beams arranged in a three-dimensional sieve-like structure. The beams are lined with a monolayer of cells called trabecular cells. Schlemm's canal is adjacent to the trabecular meshwork. The outer wall of the trabecular meshwork coincides with the inner wall of Schlemm's canal. The aqueous humor travels through the spaces between the trabecular beams, across the inner wall of Schlemm's canal into the canal, through a series of about twenty-five collecting channels that drain from Schlemm's canal and into the episcleral venous system. In a normal situation, aqueous production is equal to aqueous outflow and intraocular pressure remains fairly constant in the range of 10 to 20 mmHg. In primary open angle glaucoma, which is the most common form of glaucoma, the abnormal resistance is believed to be along the outer aspect of the trabecular meshwork and the inner wall of Schlemm's canal. It is believed that an abnormal metabolism of the trabecular cells leads to an excessive buildup of extracellular materials or a buildup of abnormally “stiff” materials in this area. Thus, dilatory means for uncollapsing these tissues become desirable. Histopathology of glaucoma eyes also demonstrates a collapse of Schlemm's canal. Primary open angle glaucoma accounts for approximately eighty-five percent of all glaucoma. Other forms of glaucoma (such as angle closure glaucoma and secondary glaucoma) also involve decreased outflow through the canalicular pathway, but the increased resistance is from other causes such as mechanical blockage, inflammatory debris, cellular blockage, etc.

The disease is estimated to affect up to 4% of all adults over 40 years old (Leske, M. C. et al., Amer. J. Epidemiol. 113:1843-1846 (1986); Bengtsson, B., Br. J. Ophthamol. 73:483-487 (1989); Strong, N. P., Ophthal. Physiol. Opt. 12:3-7 (1992)). Moreover, up to 6% of those 75 years or older are also afflicted with the disease (Strong, N. P., Ophthal. Physiol. Opt. 12:3-7 (1992)).

The increased (IOP) or intraocular pressure is a readily measurable sign of glaucoma. Thus, the diagnosis of the disease is largely screened for by measuring intraocular pressure (tonometry) (Strong, N. P., Ophthal, Physiol. Opt. 12:3-7 (1992), Greve, M. et al., Can. J. Ophthamol. 28:201 -206 (1993)). For more accurate measurements of severity of glaucoma in term of IOP or, additional methods, such as direct examination of the optic disk and determination of the extent of a patient's visual field loss are often conducted to improve the accuracy of diagnosis (Greve, M. et al., Can. J. Ophthamol. 28:201-206 (1993)).

Prior art treatment of glaucoma consists in lowering the intraocular pressure to a level that is tolerable for the optic nerve so that the progression of damage and visual loss is halted. Certainly, enhancing the blood flow to the optical nerve, despite the high IOP, is also a viable solution. Treatment of glaucoma is either device-based (implants, prostheses) or drug-based. The drugs currently used for glaucoma fall into classes according to their mechanism of action. They are beta-blockers (such as timolol, betaxolol, levobunolol), sympathomimetics (such as epinephrine and dipivephrine), parasympathomimetics or miotics (such as pilocarpine and acetylcholine) and carbonic anhydrase inhibitors (such as acetazolamide and dichlorphenamide) and beta.-adrenergic agonists such as dorzolamide hydrochloridetimolol maleate which is the combination of a topical carbonic anhydrase inhibitor and a topical beta-adrenergic receptor blocking agent (COSOPT_{RTM}). It is well recognized that regulation of aqueous humor outflow through the trabecular meshwork is critically important for maintenance of an appropriate intra-ocular pressure, and that in disease states such as ocular hypertension and glaucoma, this regulation appears to be defective. For instance, historically U.S. Pat. No. 4,757,089 teaches a method for increasing aqueous humor outflow by topical or intracameral administration of ethacrynic acid, or an analog, to treat glaucoma. There are many more patents along the same line of approach, which will not be discussed here. More recently other classes of drugs, i.e. the calcium blocking agents as well as dilatory agents such as prostaglandin F (latanoprost, XLATAN^{RTM}) have been introduced. The former, also known as “calcium entry blockers” or “calcium antagonists”, are currently used as vasodilators and in the treatment of cardiac affections. For such indications, the most widespread calcium antagonists are, e.g., nifedipine, diltiazem and verapamil. For the latter, the reader is referred to U.S. Pat. No. 6,353,000 to Sallee , et al. entitled “11-halo prostaglandins for the treatment of glaucoma or ocular hypertension” which basically describes a method of treating glaucoma or ocular hypertension in a patient, which comprises administering to the patient a pharmaceutically effective amount of a compound of 11-halo prostaglandins, and the U.S. Pat. No. 6,344,485B1 to Cameron entitled “method for treating glaucoma” which basically covers methods of using prostaglandin agonists for the reduction of intraocular pressure, and accordingly glaucoma and U.S. Pat. No. 6,329,426 to Ueno entitled “method for treating ocular hypertension of glaucoma” which describes a combined administration of a non-FP receptor agonist type prostaglandin compound and a FP-receptor type prostaglandin compound.

The current topical treatments of glaucoma suffer from a number of side effects, which justifies searching for other types of topical treatment for glaucoma. Recently, we have concluded that certain combination of a nitric oxide (NO) releasing agent or donor, such as nitrovasodilators like nitroglycerin, L-arginine, isosorbide dinitrate, nitroprusside, or pyrimidine (also known as minoxidil) and the cyclic guanosine 3′5′-monophosphate (cGMP) specific phosphodiesterase type 5 (PDE5) inhibitor such as TRM sildenafil citrate (Viagra^{TRM}) in an ophthalmologically acceptable solution mix, can cause ocular hypotensive effects in human eye and lower the IOP without any observable side effects. The invention considers an active highly reactive free radical of Nitric Oxide. For the purposes of this patent application “nitric oxide”, “NO” and “NO ⋄ ” will represent it, throughout.

The prior art does not describe nor suggest the combination of the present invention for glaucoma treatment. Therefore, the aim of the present invention is to describe a new topical drug (ointment or eye drop) for treating glaucoma or ocular hypertension in a patient, which comprises a mixture of a nitric oxide (NO) donor and a cyclic guanosine 3′,5′-monophosphate (cGMP) specific phosphodiesterase type 5 (PDE5) inhibitor such as sildenafil citrate (Viagra^{RTM}) in an ophthalmologically acceptable solution mix.

### SUMMARY OF INVENTION

The present invention describes a finding that combination of nitric oxide (NO) releasing agents such as nitroglycerin, L-arginine, isosorbide dinitrate, sodium nitroprusside (sodium nitroferricyanide), or pyrimidine (also known as Minoxidil or Rogaine^{RTM}), or alternatively 2,4-Diamino-6-piperidinopyrimidine 3-N-oxide and a cyclic guanosine 3′,5′-monophosphate (cGMP) specific phosphodiesterase type 5 (PDE5) inhibitor such as sildenafil citrate (Viagra^{RTM}) or 1-[[3-(6,7-dihydro-1methyl-7-oxo-3-propyl-1-H-pyrazolo[4,3-d]pyrimidin 5-yl)-4-ethoxyphenyl]sulfonyl]-4 methylpiperazine citrate, in an ophthalmologically acceptable solution mix, when administered orally or topically in proper concentration, works synergistically to promote systemic vascular relaxation, enhanced blood flow to the optic nerve, dilation of the trabecular meshwork, the Schlemm's canal and uveoscleral outflow channel tissues, enhanced aqueous humor drainage and thus lowered intraocular pressure (IOP) in mammalian eye. The basic mechanism at work here is that mammalian trabecular meshwork (TM) cells, i.e., bovine and human, can respond to nitrovasodilators in a much more pronounced manner if the systemic vascular relaxation and dilation of the trabecular meshwork, the Schlemm's canal and uveoscleral outflow track tissues due to the presence of incipient cyclic guanosine 3′,5′-monophosphate (cGMP) is not impeded by the presence of cGMP specific degrading enzyme of phosphodiesterase type 5 (PDE5). Therefore, as a first step in solving this problem a chemical inhibitor of phosphodiesterase type 5 (PDE5) must be present. In addition to such presence, however, the synthesis of smooth muscle relaxant guanosine 3′,5′-monophosphate (cGMP) must be preceded by the presence of activated enzyme guanylate cyclase (sGC) which then causes the synthesis of guanosine monophosphate (cGMP) to take place. However, the enzyme guanylate cyclase (sGC) needs to be activated by the presence of an active nitric oxide radical (NO) or chemicals that are capable of releasing NO in such tissues. Thus, the presence of a NO releasing agents such as nitrovasodilators will be necessary. The mixture can synthesize NO in areas that cannot themselves produce it, thereby promoting systemic vascular relaxation and dilation in order to reduce ocular hypertension.

### DETAILED DESCRIPTION

The present invention provides a method for increasing aqueous humor outflow in the eye of a human or other mammal by administration to the eye of an effective amount of a compound that substantially enhances the production of cyclic guanosine 3′,5′-monophosphate (cGMP) and simultaneously inhibits the production of cGMP specific phosphodiesterase type 5 (PDE5). The output-increasing compound can be administered to the eye topically in the from of an ointment or an eye drop and is therapeutically useful, in reducing the intra-ocular pressure in prevention ocular hypertension and glaucoma. The present invention proposes a topical drug for glaucoma which comprises a mixture of a nitric oxide (NO) donor such as nitrovasodilators like minoxidil, nitroglycerin, L-arginine, isosorbide dinitrate, or nitroprusside, and a cyclic guanosine 3′,5′-monophosphate (cGMP) specific phosphodiesterase type 5 (PDE5) inhibitor such as sildenafil citrate (Viagra^{RTM}) in an ophthalmologically acceptable solution mix. In this manner there will be increased blood circulation to the optic nerve and the ocular hypotensive effect of the combined compounds is enhanced synergistically.

Nitric oxide is a gaseous molecule produced in the body through the enzymatic degradation of L-arginine. “Nitric oxide donor compound” means any compound (including small molecules, polymers, etc.) that releases nitric oxide or which acts as a substrate leading to the formation of nitric oxide. A wide variety of nitric oxide donor compounds are available for the release/production of nitric oxide, including the following:

Organic nitrates such as nitroglycerine.

O-nitrosylated compounds also known as O-nitroso compounds or in some cases organic nitrites).

S-nitrosylated compounds also known as S-nitroso compounds or S-nitrosothiols compounds such as glutathione, S-nitrosylated derivatives of captopril, Snitrosylated-proteins/peptides, S-nitrosylated oligosaccharides and polysaccharides.

Nonoates compounds such as piperazines 2 and diazeniumdiolates.

Inorganic nitroso compounds such as sodium nitroprusside.

Sydnonimines.

L-arginine (which does not release NO directly, but rather is an enzyme substrate which leads to the formation of nitric oxide in vivo).

1,3-(nitrooxymethyl)phenyl 2-hydroxybenzoate

isosorbide dinitrateand pyrimidine (also known as Minoxidil or Rogaine^{RTM})

A substance released by the endothelium, “endothelium derived relaxing factor” (EDRF) is now known to be nitric oxide (NO) or a compound, which liberates NO. This substance relaxes vascular smooth muscle, inhibits platelet aggregation, inhibits mitogenesis and proliferation of cultured vascular smooth muscle, and leukocyte adherence. NO may have other effects, either direct or indirect, on the various cells associated with vascular wails and degenerative diseases of the vessel.

Thus, the presence of NO releasing agents such as nitrovasodilators will be necessary.

The mixture can synthesize NO in areas that cannot themselves produce it, thereby promoting systemic vascular relaxation and dilation in order to reduce ocular hypertension. The release of NO stimulates the activation of an enzyme synthesis of guanosine 3′5′-cyclic monophosphate, (cGMP) in a target cell by directly activating the soluble isoform of enzyme guanylate cyclase (sGC). NO then activates the enzyme guanylate cyclase, which results in increased levels of synthesis of cyclic guanosine monophosphate (cGMP), which should escape degradation by phosphodiesterase, type 5 (PDE5) enzyme. Thus, the presence of a guanosine 3′,5′-monophosphate (cGMP) specific phosphodiesterase type 5 (PDE5) inhibitor such as sildenafil citrate (Viagra^{RTM}) which is designated chemically as 1-[[3-(6,7-dihydro-1-methyl-7-oxo-3-propyl-1-H-pyrazolo[4,3-d]pyrimidin-5-yl)-4-ethoxyphenyl]sulfonyl]-4 methylpiperazine citrate, is necessary. Some suitable cGMP PDE5 inhibitors for the use according to the present invention include:

1 -[[3-(6,7dihydro-1-methyl-7-oxo-3-propyl-1H-pyrazolo[4,3-d]pyrimidin-5-yl)-4 ethoxyphenyl]sulphonyl]-4-methylpiperazine (sildenafil);

3-ethyl-S-[5-(4-ethylpiperazin-1-ylsulphonyl)-2-(2-methoxyethoxy)pyridin-3-yl]-2(pyridin-2-yl)methyl-2,6-dihydro-7H-pyrazolo[4,3-d]pyrimidin-7-one;

1-{6-ethoxy-5-[3-ethyl-6,7-dihydro-2-(2-methoxyethyl)-7-oxo-2H-pyrazolo[4,3-d]pyrimidin-5-yl]-3-pyridylsulphonyl}-4-ethylpiperazine;

5-(5-Acetyl-2-propoxy-3-pyridinyl)-3-ethyl-2-(1 -isopropyl-3-azetidinyl)-2,6-dihydro-7H-pyrazolo[4,3-d]pyrimidin-7-one;

5-(5-Acetyl-2-butoxy-3-pyridinyl)-3-ethyl-2-(1 -ethyl-3-azetidinyl)-2,6dihydro-7H-pyrazolo[4,3-d]pyrimidin-7-one;

The suitability of any chosen cGMP PDE5 inhibitor can be readily determined by evaluation of its potency and selectivity by evaluation of its toxicity, absorption, metabolism, pharmacokinetics, etc in accordance with standard pharmaceutical practice. The Preferred cGMP PDE5 inhibitor here is Sildenafil citrate of Pfizer also known as Viagra^{RTM}.

Thus, the present invention provides a method for increasing blood flow to the optic nerve and increasing aqueous humor outflow in the eye of a human or other mammals by administration to the eye of an effective amount of a compound that substantially enhances the production of cyclic guanosine 3′5′-monophosphate (cGMP) and simultaneously inhibits the production of cGMP specific phosphodiesterase type 5 (PDE5) which tend to impede the production of cyclic guanosine 3′5′-monophosphate (cGMP). The compound of the invention can be administered to the eye topically in the form of an ophthalmic ointment or eye drop. Note that said topical ophthalmic solution can be in the form of an aqueous solution or suspension, or in the form a gel, an ointment or a cream in a pharmaceutically acceptable ophthalmic vehicle, or in the form of an erodible ocular insert or of a “reservoir” system with active polymer membrane (Shahinpoor et al, Ionic PolymerMetal Composites I. Fundamentals, (Review Paper), Smart Materials and Structures Int. J., Vol. 10, pp. 819-833 (2001)), to be placed in the conjunctival sac or other drug releasing means with remote control capability.

Mammalian trabecular meshwork (TM) cells, i.e., bovine and human, can respond to NO releasing agents such as nitrovasodilators in a much more pronounced manner if the systemic vascular relaxation and dilation of the trabecular meshwork, the Schlemm's canal and uveoscleral outflow track tissues due to the presence of incipient cyclic guanosine 3′,5′-monophosphate (cGMP) is not impeded by the presence of cGMP specific degrading enzyme of phosphodiesterase type 5 (PDE5). Therefore, as a first step in solving this problem a chemical inhibitor of phosphodiesterase type 5 (PDE5) must be present. In addition to such presence, however, the synthesis of smooth muscle relaxant guanosine 3′,5′-monophosphate (cGMP) must be preceded by the presence of activated enzyme guanylate cyclase (sGC) which then causes the synthesis of guanosine monophosphate (cGMP) to take place. However, the enzyme guanylate cyclase (sGC) needs to be activated by the presence of an active nitric oxide radical (NO) or chemicals that are capable of releasing NO in such tissues. Thus, the presence of NO releasing agents such as nitrovasodilators will be necessary. The mixture can synthesize NO in areas that cannot themselves produce it, thereby promoting systemic vascular relaxation and dilation in order to reduce ocular hypertension. The release of NO stimulates the synthesis of guanosine 3′5′-cyclic monophosphate, (cGMP) in a target cell by directly activating the soluble isoform of enzyme guanylate cyclase (sGC). NO then activates the enzyme guanylate cyclase, which results in increased levels of synthesis of cyclic guanosine monophosphate (cGMP) which escapes degradation by phosphodiesterase type 5 (PDE5) enzyme, in the presence of a guanosine monophosphate (cGMP) specific phosphodiesterase type 5 (PDE5) inhibitor such as sildenafil citrate (Viagra^{RTM}) which is designated chemically as 1-[[3-(6,7-dihydro-1-methyl-7-oxo-3-propyl-1H-pyrazolo[4,3-d]pyrimidin-5-yl)-4-ethoxyphenyl]sulfonyl]-4methylpiperazine citrate.

Historically, nitrovasodilators have been found to increase outflow facility and decrease intra-ocular pressure in monkey eye (Schuman, et al., Exp. Eye Res., 58:99-105, 1994). However, the combined effect of nitrovasodilators to promote the release of nitric oxide (NO) to activate the enzyme guanylate cyclase (sGC) for the increased level of synthesis of guanosine 3′5′-cyclic monophosphate, (cGMP) in the presence of cGMP specific PDE5 inhibitors in a concurrent fashion to lower IOP and treat glaucoma is a novel concept that has not been yet discussed in the pertinent literature.

In addition to nitric oxide (NO) releasing agents such as nitroglycerin or C3H5N3O9, sodium nitroprusside (sodium nitroferricyanide) or Na2 Fe (CN)5NO-2H₂O, pyrimidine (also known as Minoxidil or Rogaine^{RTM}), or C9H15N5O or alternatively 2,4-Diamino 6-piperidinopyrimidine 3-N-oxide, (Ignarro et al., J. Pharmacol. Exp. Ther., 218, 739-749 (1981); Ignarro, Annu. Rev. Pharmacol. Toxicol., 30, 535-560 (1990); Kruszyna et al., Toxicol. Apol. Pharmacol., 91, 429-438 (1987); Wilcox et al., Chem. Res. Toxicol., 3, 71-76 (1990)), are other NO releasing compounds of interest to the present invention. The reader is referred to U.S. Pat. No. 6,379,660 to Saavedra, et al. Entitled “Nitric oxide-releasing 1-[(2 carboxylato) pyrrolidin-1-yl]diazen-1-ium-1,2-diolates and composition comprising same” which discusses a polymeric composition capable of releasing nitric oxide under physiological conditions which includes a biopolymer, such as a peptide, polypeptide, protein, oligonucleotide or nucleic acid, to which is bound a nitric oxide-releasing functional group and pharmaceutical compositions comprising the polymeric composition, U.S. Pat. No. 6,391,895 to Towart , et al. , entitled “Nitric oxide releasing chelating agents and their therapeutic use”, that discusses chelating agents, in particular dipyridoxyl and aminopolycarboxylic acid based chelating agents, and their metal chelates, when linked directly or indirectly to at least one nitric oxide releasing moiety, or when use in combination with nitric oxide or a nitric oxide releasing moiety.

Accordingly, the present invention provides:

an ophthalmic composition containing a combination of NO releasing agents and cGMP-PDE5 inhibitors;

an ocular hypotensive composition containing NO releasing agents and cGMP-PDE5 inhibitors;

the ocular vasodilatory composition containing NO releasing agents and cGMP-PDE5 inhibitors;

a method for preventing and treating glaucoma, comprising administering a pharmaceutically effective amount of a Compound containing NO releasing agents and cGMP-PDE5 inhibitors;

a method for lowering intraocular pressure, comprising administering a pharmaceutically effective amount of a Compound containing NO releasing agents and cGMP-PDE5 inhibitors;

The foregoing invention has been described in some detail by way of illustration and example for purposes of clarity of understanding to make it readily apparent to those of ordinary skill in the related art that certain changes and modifications may be made thereto without departing from the spirit or scope of the appended claims.

1- Method for lowering ocular hypertension, comprising administering, to a patient in need thereof, a topical ophthalmic eye drop or ointment containing no releasing agents and cGMP-PDE5 inhibitors;

2- method according to claim 1, wherein said topical ophthalmic eye drop or ointment is in the form of an aqueous solution or suspension, or in the form a gel, or a cream in a pharmaceutically acceptable ophthalmic vehicle, or in the form of an erodible ocular insert or of a “reservoir” system with a polymer membrane or a gel to be placed in the conjunctival sac.

3- Method according to claim 1, wherein the No releasing agent in said ophthalmic medicament is Organic nitrates such as nitroglycerine.

4- Method according to claim 1, wherein the No releasing agent in said ophthalmic medicament is O-nitrosylated compounds also known as O-nitroso compounds or in some cases organic nitrites.

5- Method according to claim 1, wherein the No releasing agent in said ophthalmic medicament is S-nitrosylated compounds also known as S-nitroso compounds or S-nitrosothiols compounds such as glutathione.

6- Method according to claim 1, wherein the No releasing agent in said ophthalmic medicament is S-nitrosylated derivatives of captopril.

7- Method according to claim 1, wherein the No releasing agent in said ophthalmic medicament is S-nitrosylated-proteins/peptides.

8- Method according to claim 1, wherein the No releasing agent in said ophthalmic medicament is S-nitrosylated oligosaccharides and polysaccharides.

9- Method according to claim 1, wherein the No releasing agent in said ophthalmic medicament is Nonoates compounds such as piperazines 2 and diazeniumdiolates.

10- Method according to claim 1, wherein the No releasing agent in said ophthalmic medicament is Inorganic nitroso compounds such as sodium nitroprusside.

11- Method according to claim 1, wherein the No releasing agent in said ophthalmic medicament is Sydnonimines.

12- Method according to claim 1, wherein the No releasing agent in said ophthalmic medicament is L-arginine (which does not release NO directly, but rather is an enzyme substrate which leads to the formation of nitric oxide in vivo).

13- Method according to claim 1, wherein the No releasing agent in said ophthalmic medicament is 1,3-(nitrooxymethyl)phenyl 2-hydroxybenzoate isosorbide dinitrate.

14- Method according to claim 1, wherein the No releasing agent in said ophthalmic medicament is pyrimidine (also known as Minoxidil or Rogaine^{RTM}).

15- Method according to claim 1, wherein the cGMP specific PDE-5 inhibitor in said ophthalmic solution is (sildenafil) also known as 1-[[3-(6,7-dihydro-1 methyl-7-oxo-3-propyl-1H-pyrazolo[4,3-d]pyrimidin-5-yl)-4 ethoxyphenyl]sulphonyl]-4-methylpiperazine.

16- Method according to claim 1, wherein the cGMP specific PDE-5 inhibitor in said ophthalmic solution is sildenafil citrate, (Viagra^{RTM}) also known as 1-[[3(6,7-dihydro-1-methyl-7-oxo-3-propyl-1H-pyrazolo[4,3-d]pyrimidin-5-yl)4 ethoxyphenyl]sulphonyl]-4-methylpiperazine citrate.

17- Method according to claim 1, wherein the cGMP specific PDE-5 inhibitor in said ophthalmic solution is 3-ethyl-5-[5-(4-ethylpiperazin-1-ylsulphonyl)-2n-propoxyphenyl]-2 -(pyridin-2-yl)methyl-2,6-dihydro-7H-pyrazolo[4,3-d]pyrimidin-7-one.

18- Method according to claim 1, wherein the cGMP specific PDE-5 inhibitor in said ophthalmic solution is 1-{6-ethoxy-5-[3-ethyl-6,7-dihydro-2-(2-methoxyethyl)-7-oxo-2H-pyrazolo[4,3-d]pyrimidin-5yl]-3 pyridylsulphonyl}-4-ethylpiperazine.

19- Method according to claim 1, wherein said topical ophthalmic solution is in the form of an aqueous solution and further contains one or more tonicity adjusting agents, one or more buffers and one or more antioxidants.

20- Method according to claim 1, wherein said topical ophthalmic solution further contains one or more antimicrobial agents.

21- The composition according to claim 1, wherein said dose is in pill form for oral administration.

22- The method according to claim 1, wherein said topical ophthalmic solution further contains one or more combinations of NO donors and cGMP PDE5 inhibitors.

23- The method according to claim 1, wherein said topical ophthalmic solution further contains one or more weight percentage combinations of NO donors and cGMP PDE5 inhibitors.
